(19) United States
(12) Patent Application Publication
(10) Pub. No.: US 2011/0130836 A1
(43) Pub. Date: Jun. 2, 2011

(54) Compositions and Methods for Use of Scar Tissue in Repair of Weight Bearing Surfaces

(75) Inventors: Jeffrey John Thramann, Niwot, CO (US); Keyvan Behnam, Simi Valley, CA (US)

(73) Assignee: LANX, INC., Broomfield, CO (US)

(21) Appl. No.: 12/861,727

(22) Filed: Aug. 23, 2010

Related U.S. Application Data
(63) Continuation-in-part of application No. PCT/US2009/034632, filed on Feb. 20, 2009.
(60) Provisional application No. 61/030,348, filed on Feb. 21, 2008.

(51) Int. Cl. A61F 2/44; A61B 17/60; A61F 2/30

(52) U.S. Cl. 623/17.11; 606/90; 623/18.11

(57) ABSTRACT

Compositions and methods are provided for weight bearing surface, i.e., intervertebral disc (“disc”), between spinous processes and articular cartilage, repair. Compositions include fibrosis inducing agents for facilitating fibrosis in or on the site in need of repair in order to form fibrotic connective tissue. In addition, methods are provided for distracting the appropriate disc space during treatment of a disc in need of repair, and in particular treatment of the disc in need of repair with a fibrosis inducing agent. Further, methods and compositions are provided for inducing or forming fibrotic tissue at a bone surface having damaged cartilage or in a joint in need of repair.

### TECHNICAL FIELD

The present invention relates to the use of fibrotic tissue in various weight bearing tissue repair, and in particular to forming fibrotic tissue in intervertebral discs in need of repair, between adjacent spinous processes in need of support and repair and in replacement of articular cartilage or articular cartilage surfaces in need of repair, or so as to act as an autologous replacement of damaged or degenerated tissue.

### BACKGROUND

Articular cartilage covers the surfaces of all diarthrodial joints in the body. Cartilage damage and joint disrepair is a common and increasingly important issue, especially in the elderly. Repair of joint cartilage, for example, often requires replacement of the entire joint, e.g., knee replacement, artificial injection of a lubricating fluid(s), e.g., phospholipid compositions, and/or cartilage repair and re-growth, e.g., chondrocyte or stem cell based repair therapies. In each situation, the repair is performed in order to minimize scarring at the site of repair and maximize function between the corresponding bone surfaces.

Intervertebral disc (herein after “disc”) are pad like structures found interposed between vertebrae within the spine. Each disc functions to maintain the basic integrity of the spine, i.e., provide proper physical and neural spacing and contact between the vertebrae end-plates of the spine, provide pivot points for the spine to bend and twist, and provide a shock absorber material to carry the axial load on a body when the body is in an up right position.

There are twenty three discs in the human spine, each composed of three basic zones: the nucleus pulposus, the annulus fibrosis and the lamellae. Within these three zones several main components provide for the disc functions described above, these components include proteoglycans, collagen and water.

With wear and tear, discs can degrade or become damaged (typically in individuals over 35) leading to pain and, in some cases, loss of function. Degenerated or damaged disc are typically treated by ameliorating the symptoms of the damage, including pain medications, physical therapy and rest. However, in some instances a more aggressive treatment regime is required, including surgical procedures focused on removal of part or all of the damaged disc, fusion procedures to affix the adjoining vertebrae, posterior stabilization procedures, and replacement of the disc with artificial discs.

To date there has not been an adequate procedure directed at disc repair that provides a long term flexible, hydrated replacement having similar properties to an undamaged disc.

As such, for both disc and articular cartilage repair there is no adequate method for replacement.

The present invention is directed toward overcoming one or more of the problems discussed above.

### SUMMARY

The present invention provides compositions and methods for inducing fibrotic tissue at damaged tissue sites and particularly at weight bearing tissue sites. The induced fibrotic tissue of the invention provides substituted or replaced functionality at the damage site in the form of a flexible, hydrated and resilient scar.

In one aspect, the present invention provides compositions and methods for inducing and/or forming fibrotic tissue in intervertebral discs in need of repair.

One embodiment provides fibrosis inducing agents for use in inducing or forming fibrotic tissue in an intervertebral disc in need of repair. Fibrosis inducing agents in accordance with embodiments herein include: agents that create an acidic environment in the disc in need of repair; agents that create a basic environment in the disc in need of repair; agents that induce fibroblast proliferation and/or recruitment into a disc in need of repair; and agents that irritate sites within the disc in need of repair.

Other aspects of this embodiment include fibrosis inducing agents as part of pharmaceutical compositions used in the treatment of patients having a disc in need of repair.

Another embodiment provides methods for distracting the disc space relative to the disc in need of repair during or after treatment of the disc in need of repair with a fibrosis inducing agent(s). Distraction of the disc space enhances the capacity of the fibrosis inducing agent to induce and/or form fibrosis in the disc in need of repair. Aspects of this embodiment include: the use of intervertebral disc distraction techniques; use of re-absorbable material to stabilize and expand the disc space; use of permanent fixation techniques for distraction of the disc space; and use of the fenstrom ball to distract the disc space.

Other embodiments include cell based therapies, e.g., stem cells, fibroblasts, etc, being delivered into the disc in need of repair in conjunction with treatment of the disc with fibrosis inducing agent(s) or treatment of the disc with fibrosis inducing agent and distraction of the disc space.

In addition, embodiments herein provide methods for treatment of a disc in need of repair with fibrosis inducing agents in combination with angiogenesis inducing agents as well as treatment of a disc in need of repair with fibrosis inducing agents and angiogenesis inducing agents in combination with distraction of the appropriate disc space.

In another aspect of the invention, compositions and methods are provided for inducing and/or forming fibrotic tissue between adjacent spinous processes in need of support there-between. For example, compositions and methods for inducing fibrotic/scar tissue formation between spinous processes instead of or in combination with interspinous process implants or spacers.

One embodiment provides fibrosis inducing agents for use in inducing or forming fibrotic tissue between adjacent spinous processes in need of support there-between. Fibrosis inducing agents include agents that create an acidic environment in the space between adjacent spinous processes; agents that create a basic environment between adjacent spinous processes; agents that induce fibroblast proliferation and/or recruitment into the space between adjacent spinous processes; and agents that irritate sites between adjacent spinous processes. Fibrous inducing agents can be contacted to the site between spinous processes with or without a scaffolding material. Typical scaffolding materials for use herein include: resorbable polymers, collagen, cotton, and other like materials. In alternative embodiments, the fibrous inducing agents are contacted to the site between spinous processes in combination with fibronectin or other chemoattractant agents for fibroblasts.

In yet another aspect of the invention, compositions and methods are provided for inducing and/or forming fibrotic tissue at or on bone surfaces in joints where articular cartilage is in need of repair.

One embodiment provides fibrosis inducing agents for use in inducing or forming fibrotic tissue at a bone surface lacking or devoid of cartilage or at a bone surface having damaged cartilage. Fibrosis inducing agents in accordance with embodiments herein include: agents that create an acidic environment in or on the bone in need of repair; agents that create a basic environment in or on the bone in need of repair; agents that induce fibroblast proliferation and/or recruitment into the joint space in need of repair; and agents that irritate sites on or around the bone in need of repair. Note that in some embodiments the compositions and methods are utilized to form fibrotic tissue on bone surfaces opposite each other in the joint in need of repair. In addition, each of the above compositions can be used within or on damaged cartilage on a bone surface in a joint in need of repair or in conjunction with implants or scaffolding needed to support development of sufficient fibrotic tissue. Finally, as above, fibrosis inducing agents used to repair damaged articular cartilage can be combined with fibronectin or other chemoattractant agents for fibroblasts.

Some embodiments provide for the repair and/or replacement of at least part of a patient's spinal disc with fibrous tissue formed at least in part using a partially or fully inactivated demineralized bone matrix (DBM). In some embodiments the inactivated DBM is inserted with a spinal implant, such as an interbody spacer positioned between two adjacent vertebrae. The interbody spacer may operate to help maintain or restore a desired spacing or height between two adjacent vertebrae, while the DBM promotes fibrous tissue formation in the disc space. The interbody spacer and fibrous tissue combination helps provide for at least some motion at the disc site.

Further embodiments provide compositions and methods for inducing fibrous and/or fibrocartilaginous tissue formation while modulating the osteogenic potential of demineralized bone matrix (DBM). In some embodiments, the osteogenic potential of DBM is modulated through heat inactivation, such as through heat inactivation of BMP-2. In other embodiments, the osteogenic potential of DBM is modulated through chemical treatment, such as through chemical inactivation or chemical extraction of one or more BMPs. In still other embodiments, the osteogenic potential of DBM is modulated through treatment of DBM with an antagonist or antibody, such as an antagonist or antibody to BMP-2. An illustrative BMP-2 antagonist is SPP-24.

Further embodiments provide kits for inducing fibrous and/or fibrocartilaginous tissue formation while modulating the osteogenic potential of DBM. Kits include a scaffold along with instructions for performing the method of inducing fibrous and/or fibrocartilaginous tissue formation while modulating the osteogenic potential of DBM.

Still further embodiments provide systems for inducing fibrocartilaginous tissue formation while modulating the osteogenic potential of DBM. Systems include a scaffold such as an interbody spacer in combination with a bioactive component to induce fibrocartilaginous tissue formation.

These and various other features and advantages of the invention will be apparent from a reading of the following detailed description and a review of the appended claims.

### BRIEF DESCRIPTION OF THE DRAWINGS

FIG. 1 shows the points of IVD treatment along the spine of the sheep, according to an embodiment. Each animal receives one control and one experimental treatment in separate discs, with one unoperated level between treated discs.

### DETAILED DESCRIPTION

Methods and compositions are provided for inducing fibrotic tissue at damaged tissue sites and particularly at weight bearing tissue sites. The induced fibrotic tissue of the invention provides substituted or replaced functionality at the damage site in the form of a flexible, hydrated and resilient scar. Although the present invention provides methods and compositions for inducing fibrotic tissue at any weight bearing site, three are discussed in detail: atricular cartilage, intervertebral disc and between adjacent spinous processes. The discussion is, however, not meant to limit the scope of the invention beyond weight bearing sites, but rather to provide illustrative examples of the unexpected and surprising utility of using fibrotic tissue as a source of repair at weight bearing sites.

In one aspect, methods and compositions are provided for intervertebral disc (disc herein) repair. Methods and compositions described herein include contacting an intervertebral disc in need of repair with a fibrosis inducing agent. The fibrosis inducing agent facilitates fibrous connective tissue development in the disc by replacement of inflexible damaged disc with flexible fibrous connective tissue. Replacement fibrous connective tissue provides a flexible and hydrated tissue that has similar characteristics to an undamaged disc; the fibrous disc does not require further repair procedures. For purposes of the disclosure herein “disc(s) in need of repair” include discs having: degenerative disc disease, herniated or ruptured disc, annular tears, or other like damage. Discs in need of repair are typically in human patients, although the methods and compositions could be performed on other vertebrates, for example by a veterinarian on a family pet.

In one embodiment of the invention methods are provided for contacting a site in a disc in need of repair with a fibrous inducing agent. Contact can be accomplished one or more times with, for example, an amount of fibrous inducing agent to facilitate an acidic environment, an amount of fibrous inducing agent to facilitate a basic environment, an amount of fibrous inducing agent to facilitate an “irritated” environment, an amount of fibrous inducing agent for inducing fibroblast recruitment, and/or an amount of fibrous inducing agent for inducing stem cell recruitment.

In other embodiments of the invention, methods are provided for stabilizing or maintaining the space height of adjacent vertebrae that constrain the disc in need of repair. Compositions and methods of the invention thereby relieve pressure on the damaged disc during fibrous connective tissue inducement and development.

In still further embodiments of the invention, methods are provided for facilitating blood flow to a disc in need of repair that has been (or will be) treated with fibrous inducing agent(s). The methods optimize the conditions required to induce and obtain fibrous connective tissue replacement in a disc in need of repair.

In some cases, discs in need of repair are treated with fibrous inducing agent(s) while blood flow to the disc has been facilitated and while the disc space has been distracted or stabilized. Any combination of compositions and methods described herein are within the scope of the present invention.

In another aspect, methods and compositions are provided herein for inducing and/or forming fibrotic tissue between adjacent spinous processes in need of support there-between. For example, compositions and methods for inducing fibrotic/scar tissue formation between spinous processes instead of or in combination with interspinous process implants or spacers.

In one embodiment of the invention methods are provided for contacting a site between adjacent spinous processes in need of support with a fibrous inducing agent. Contact can be accomplished one or more times with, for example, an amount of fibrous inducing agent to facilitate an acidic environment, an amount of fibrous inducing agent to facilitate a basic environment, an amount of fibrous inducing agent to facilitate an irritated environment, an amount of fibrous inducing agent for inducing fibroblast recruitment, and/or an amount of fibrous inducing agent for inducing stem cell recruitment. Fibrous inducing agents can be contacted to the site in combination with implant and/or spacer materials to facilitate fibrous connective tissue and overall support between the spinous processes in need of support/repair.

Further aspects of the invention include methods and compositions for replacement and repair of articular cartilage defects, for example on bone surfaces within the knee joint or hip joint. The fibrosis inducing agent facilitates fibrous connective tissue development on bone surface or within and on damaged cartilage with flexible fibrous connective tissue. As with the disc replacement embodiment, the fibrous connective tissue provides a flexible and hydrated tissue having similar characteristics to undamaged cartilage.

In one embodiment of the invention methods are provided for contacting a site in a joint in need of repair with a fibrous inducing agent. Contact can be accomplished one or more times with, for example, an amount of fibrous inducing agent to facilitate an acidic environment, an amount of fibrous inducing agent to facilitate a basic environment, an amount of fibrous inducing agent to facilitate an irritated environment, an amount of fibrous inducing agent for inducing fibroblast recruitment, and/or an amount of fibrous inducing agent for inducing stem cell recruitment.

Is some aspects the ends of a target joint in need of repair are ground down to facilitate scar tissue formation either contemporaneously or within a short time of administration of the fibrous inducing agent.

Further aspects include addition of implant material in conjunction with the fibrous inducing agent and/or modification of target bone surfaces. For example, a sponge or collagen implant can be combined with the fibrous inducing agent to further facilitate scar formation on the bone surfaces and within damaged cartilage. Other implant materials can be used in this manner, as would be known by one of skill in the art.

As with embodiments discussed for disc repair, joints in need of repair are treated with a combination of a fibrous inducing agent and an angiogenesis inducing agent.

### Compositions For Inducing Fibrosis

Compositions of the invention facilitate fibrous connective tissue development in weight bearing tissue in need of repair.

Compositions herein are termed “fibrous inducing agents” which refers to any agent that facilitates or accelerates fibrous connective tissue development or “scarring” in a target site, i.e., typically sites in or adjacent to disc in need of repair or joint in need of articular cartilage repair. For purposes herein, fibrous connective tissue generally refers to fibroblasts and some amount of polysaccharides, proteins (e.g., collagen) and water.

Embodiments described herein include fibrous inducing agents that create an acidic target site environment. The acidic environment provides a condition(s) in the disc or joint in need of repair that encourages fibrosis. Typical fibrous inducing agents that provide an acidic environment include polylactic acid (PLA), hyaluronic acid, and other like agents. Typical agents for creating an acidic environment are contacted to a disc site in need of repair in an amount sufficient to create an acidic environment at the site. For example, an appropriate dose of PLA is applied to a site in need of repair via direct injection.

Embodiments described herein include fibrous inducing agents that create a basic target site environment. The basic environment provides conditions in the disc in need of repair that encourages fibrosis. Typical fibrous inducing agents that provide a basic environment include bases like sodium hydroxide. Typical agents for creating a basic environment are contacted to a disc site in need of repair in an amount sufficient to create a basic environment at the site. For example, an appropriate dose of sodium hydroxide is applied to a site in need of repair via direct injection.

Embodiments described herein include fibrous inducing agents that induce fibroblast to proliferate and produce connective tissue. Agents described herein include bone morphogenetic protein 2, 4, fibroblast growth factor (FGF), PDGFRα, matrix proteins, and endothelin 1. See also Dang et al., EMBO Jr. 2004 23, 2800-2810; and Konttinen et al., Arthritis Res. 2000 2(5) 348-355, both of which are incorporated by reference in their entirety. These factors are applied in a sufficient amount to a disc site in need of repair to result in recruitment and proliferation of fibroblasts in the site in need of repair. For example, an appropriate amount of BMP-2 is applied to a site in need of repair via direct injection.

Embodiments described herein include fibrous inducing agents that irritate sites within the disc in need of repair whereby the irritation leads to or encourages fibrosis. Typical fibrosis inducing agents that provide an irritated environment include Zylous, cellulose (including cotton), atrigel, and the like. Typical agents for creating an irritated environment are contacted to a disc site in need of repair in an amount sufficient to create an irritated environment at the site. For example, an appropriate amount of Zylous is applied to a site in need of repair via direct injection.

Fibrosis inducing agent of the invention can be formulated as pharmaceutical compositions and administered to a host, preferably a mammalian host, including a human patient having a disc in need of repair, in a variety of forms adapted to the route of administration. The fibrosis inducing agent is typically combined with a pharmaceutically acceptable carrier, and may be combined with or conjugated to delivery or other like agents. Note that in some embodiments two or more different fibrosis inducing agents are combined for the treatment of a disc in need of repair. For example, BMP-2 can be combined with PLA. Combinations can be pre-combined together prior to administration or can be administered over the same course of treatment independently of each other.

Pharmaceutical compositions can be in the form of suspensions, sterile injectable preparations, or other like forms. Solutions or suspensions of the fibrosis inducing agents can be prepared in water, isotonic saline, and optionally mixed with a surfactant. Dispersions can be prepared in glycerol, liquid polyethylene, glycols, and/or triacetin. Under ordinary conditions of storage and use, these pharmaceutical compositions may contain a preservative to prevent the growth of microorganisms.

The pharmaceutical compositions of the invention suitable for injection into a disc in need of repair can include sterile, aqueous solutions or dispersions or sterile powders comprising an active ingredient which is adapted for the extemporaneous preparation of sterile injectable or infusible solutions or dispersions. In all cases, the ultimate dosage form should be sterile, fluid and stable under the conditions of manufacture and storage. Proper fluidity can be maintained, for example, by the formation of liposomes. Note that prevention of microorganisms can be accomplished by various antibacterial and antifungal agents, for example, parabens, chlorobutanol, phenol, sorbic acid, thimerosal, and the like. Prolonged absorption of the fibrosis inducing agent(s) can be brought about by the inclusion of agents such as: aluminum monostearate hydrogels and gelatin.

Sterile injectable solutions are prepared by incorporating the compounds in the appropriate solvent with various other ingredients as enumerated above and, as required, followed by filter sterilization.

In one embodiment, a fibrosis inducing agent such as a pharmaceutical composition is administered to a disc in need of repair, for example, PLA is administered to a target site in the disc in need of repair. The amount of fibrosis inducing agent needs to be sufficient to induce and maintain a patient's fibrosis response in the disc in need of repair for a period of time to result in the fibrosis of at least a portion of the disc in need of repair. In some embodiments the fibrosis inducing agent is administered until substantially the entire disc in need of repair undergoes some enhanced level of fibrosis. In particular, a disc is considered to have undergone fibrosis in response to the embodiments of the invention when a level of fibrosis is achieved that is beyond the level expected in the same site under conditions in the absence of a fibrosis inducing agent.

Fibrosis inducing agents may be administered one or more times either directly or indirectly to the disc in need of repair or directly or indirectly to the bone site or joint in need of repair. Administration can be via injection into the disc in need of repair, contacted to the disc in need of repair after a surgical procedure, released into the disc space at or adjacent the disc in need of repair, and other like procedures.

### Distraction of Disc Space At A Disc Site In Need of Repair

Methods of the invention include embodiments for distracting the disc space at a site of a disc in need of repair during the period of time required for fibrosis of the disc in need of repair. The distraction time period includes just prior to administration of a pharmaceutical composition of the invention to a disc in need of repair as well as to the period during which fibrosis occurs in the disc in need of repair.

Distraction of the disc space at a site of a disc in need of repair while fibrosis occurs facilitates the fibrosis process. In particular, distraction of the disc space alleviates pressure on the damaged disc in need of repair thereby encouraging fibroid cell growth (and consequent component production) as well as blood flow/oxygen availability to the injury site. The combination of treatment to the disc in need of repair with a fibrosis inducing agent and distraction of the disc space provides conditions for initiation and development of fibrous connective tissue. In addition, distraction of the disc space allows the space to be held open so that scar tissue fills the entire distracted disc space. In embodiments where the disc space is not distracted the treated disc may remain collapsed. Distraction and restoration of disc height therefore provides better anatomical alignment and opens both the nerve root foramen and central canal.

In one embodiment of the invention the disc space is distracted using a non-invasive distraction technique termed intervertebral disc distraction (IDD). Distraction is temporary and meant to induce negative pressure at the site of the damaged disc for periods of time sufficient to facilitate oxygen and nutrient turnover within the disc in need of repair. IDD can be performed in accordance with the invention using various non-invasive techniques including the Cor Flexion Distraction Technique, Vax-D®, or the temporary dynamic distraction technique. Temporary disc distraction is performed as known in the art for periods of time sufficient to facilitate fibrous connective tissue production in the disc in need of repair. In typical embodiments the IDD is performed once a day for 30 days and continued, if necessary, until the desired level of fibrosis is achieved in the disc in need of repair.

In another embodiment of the invention a re-absorbable material is implanted in the disc space of the disc in need of repair to temporarily expand the space while fibrosis inducing agents are administered. Typical materials for this purpose include bags or balloons developed by Spineology or re-absorbable screws to structurally stabilize adjacent vertebrae. In addition, balloons developed by Kyphon are adapted for purposes of distraction where the balloons are loaded with fibrosis inducing agents and designed to degrade over a predetermined amount of time. As such, these re-absorbable materials are implanted prior to, contemporaneous with, or just after treatment of the disc in need of repair with the fibrosis inducing agent. In some aspects the re-absorbable material is impregnated with a fibrosis inducing agent of the invention, for example, a re-absorbable bag impregnated with PLA. Re-absorbable materials used herein are used in amounts and types configured to be replaced with fibrotic tissue based on the time required for this type of tissue replacement. Re-absorption times can vary for purposes of the invention but in some embodiments re-absorption can be designed for a few days to as long as 18 months. In some embodiments re-absorption is about 3-4 months.

In another embodiment of the invention the disc space is distracted using invasive techniques. For example, techniques to hold adjacent vertebrae in place for extended periods of time while the fibrous connective tissue is formed. Techniques include pedicle screw fixation, plate fixation, flexible rod fixation, interspinous implants having shape memory, fernstrom ball implants, and other like devices. These devices remain in the patient even after the fibrosis tissue has been formed in the disc in need of repair. For purposes of illustration a patient may require distraction and the patient is getting a decompressive surgery and fusion at one level and the next level is already starting to degenerate. The decompression might be done through existing surgical techniques and the adjacent level would be protected via the fibrous at the same operation.

In one embodiment, a fernstrom ball is utilized to distract the space of the disc in need of repair. In some embodiments the fernstrom ball is modified with fibrosis inducing agent coatings that act to induce and develop fibrosis in the disc in need of repair. In one aspect the fernstrom ball is coated with PLA to provide both a mechanical spacer for disc space distraction and as a scaffold for administration of a fibrosis inducing agent in the disc in need of repair. In another aspect the fernstrom ball is coated with one or more BMPs. The coated fernstrom ball can also be implanted in conjunction with separate administration of fibrosis inducing agents to the disc in need of repair. In another aspect the fernstrom ball has a plurality of surface fenestrations and an interior reservoir for packing of fibrosis inducing agents that are released over time once the packed ball has been implanted in a disc in need of repair.

In conjunction with each of the above embodiments cell therapies can be included to facilitate the development of fibrous connective tissue in the disc in need of repair. For example, autologous or non-autologous fibroblasts or mesenchymal stem cells (MSCs) can be harvested from a patient having a disc in need of repair for implantation into the disc in need of repair. Cells, for example MSCs, can be harvested from bone marrow, peripheral blood, adipose tissue, and other like sources using techniques known in the art. In some aspects the harvested cells can be isolated and expanded using known techniques prior to implantation into the disc in need of repair using techniques known in the art.

Harvested and implanted cells are typically combined with fibrosis inducing agents and/or distraction techniques to facilitate and optimize development of a fibrotic disc in replacement of a disc in need of repair. These cells can be autologous or non-autologous. Where tissue rejection is a concern various adjuvant therapies can be administered to reduce tissue rejection as is known in the art.

In some aspects the embodiments herein can be combined with angiogenesis inducing agents. These agents are introduced to further facilitate fibrosis in the disc in need of repair by bringing additional blood nutrients to these disc sites. Typical angiogenesis inducing agents include the use of vascular endothelial growth factor (VEGF), fibroblast growth factor (FGF), angiopoietins, and matrix metalloproteinase (MMP) as well as other like agents. Note that FGF can be used for its dual capacity of inducing fibrosis within the disc in need of repair as well as for its capacity to enhance angiogenesis at the site in need of repair.

In another embodiment, mechanical disruption is performed on adjacent end plates to the disc in need of repair so as to increase access of intravascular products/agents that contribute to fibrosis into the disc in need of repair. This procedure can be combined with administration of a fibrosis inducing agent, distraction of disc space and/or introduction of angiogenesis inducing agents.

### Methods For Inducing Fibrosis In A Disc In Need of Repair

Embodiments of the invention provide methods for inducing fibrosis in a disc in need of repair. For purposes of the present disclosure “fibrosis” refers to the formation of fibrous connective tissue in a disc in need of repair. The formation of fibrous connective tissue using embodiments of the invention is enhanced as compared to formation of fibrous connective tissue in the absence of embodiments of the invention, i.e., formation of fibrous connective tissue for the disc in need of repair under normal physiologic conditions for the patient and in the absence of added fibrosis inducing agents.

In one embodiment, a health care professional or agent determines a type of fibrosis inducing agent(s); determines the requirement, if any, of distracting the disc space during administration of the fibrosis inducing agent; determines the need of MSC or fibroblast cell implantation into the disc in need of repair; and determines the need for angiogenesis inducing factors. In some embodiments the health care professional may additionally determine that mechanical disruption of an adjacent end plate may be warranted to enhance the release of intravascular products in the environment of the disc in need of repair.

The health care professional or agent then provides the desired combination of factors above to a patient having a disc in need of repair. The combination is provided or administered in a manner to facilitate fibrosis in the disc in need of repair, and therapeutic result is charted for a time sufficient to ensure the health professional's desired outcome. Outcome may be followed by symptom ablation, fibrosis detection via MRI, X-ray, or other scanning methodology or by an optical or other like technique. Additional therapeutic treatments may be required on any one patient to ensure proper fibrosis of a disc in need of repair in accordance with the present invention. Additional treatments may be the same or include new combinations to ensure that each patient obtain the best possible result.

### Methods For Inducing Fibrosis Between Adjacent Spinous Processes In Need of Support/Repair

Embodiments of the invention provide methods for inducing fibrosis between adjacent spinous processes in need of repair and/or support. For purposes of the present disclosure “fibrosis” refers to the formation of fibrous connective tissue between the adjacent spinous processes in need of support or repair. The formation of fibrous connective tissue using embodiments of the invention is enhanced as compared to formation of fibrous connective tissue in the absence of embodiments of the invention, i.e., formation of fibrous connective tissue in and around a damaged or unsupported spinous process under normal physiologic conditions for the patient and in the absence of added fibrosis inducing agents.

In one embodiment, a health care professional or agent determines a type of fibrosis inducing agent(s); determines the requirement, if any, of grinding the bone surface in the joint space during administration of the fibrosis inducing agent; determines the need of MSC or fibroblast cell implantation into the joint in need of repair; and determines the need for angiogenesis inducing factors. In some embodiments the health care professional may additionally determine that mechanical disruption of an adjacent bone surface may be warranted to enhance the release of intravascular products in the environment of the joint in need of repair. In addition the health care professional may determine that a carrier or implant material is also required, including collagen, hydrogels, reabsorbable polymer materials, and other like materials.

The health care professional or agent then provides the desired combination of factors above to a patient having a need of repair or spinous processes support. The combination is provided or administered in a manner to facilitate fibrosis, and therapeutic result is charted for a time sufficient to ensure the health professional's desired outcome. Outcome may be followed by symptom ablation, fibrosis detection via MRI, X-ray, or other scanning methodology or by an optical or other like technique. Additional therapeutic treatments may be required on any one patient to ensure proper fibrosis in accordance with the present invention. Additional treatments may be the same or include new combinations to ensure that each patient obtain the best possible result.

Methods for Inducing Fibrous and/or Fibrocartilaginous Tissue Formation while Attenuating Osteogenic Tissue Formation

Demineralized bone matrix (DBM) is capable of inducing fibrous, cartilaginous, and osseous tissue formation in an intervertebral disc space (IVD), and can be used as a bone graft extender due to its osteogenic properties. However, progression from fibrous to ossification can be undesirable. This may be the case, for example, if it is desirable to provide for some movement at the treated location. As such, provided herein are methods, compositions, systems and kits for inducing fibrous or fibrocartilaginous tissue in an IVD while attenuating osseous tissue formation. The resulting fibrocartilaginous tissue can stabilize the disc space while preserving at least some physiological motion and helping to reduce or eliminate pain associated with osseous tissue. Progression to ossification can be attenuated by at least partially, or in some cases fully, deactivating DBM.

Attenuation of osteogenic tissue formation includes either completely or partially inactivating the osteoinductive activity of DBM. The resulting scaffold can support scar formation and/or cartilage formation.

Bone matrix contains multiple growth factors, including TGF-β and osteoinductive BMPs (BMP-2, BMP-7, BMP-6, BMP-4). TGF-β stimulates scar formation, while BMPs stimulate the differentiation of cartilage and bone. It is believed that the osteoinductive signals from BMP override the ability of TGF-β to induce scar formation. Provided herein are methods and compositions for manipulation of this process by adding specific protein inhibitors of BMP activity to DBM. Exemplary inhibitors of BMP activity include, but are not limited to, alpha-2-HS glycoprotein, noggin, chordin, DAN, sclerostin, and secreted phosphoprotein 24 (SPP-24). Treatment of DBM with one or more of the BMP inhibitors would generate a resulting scaffold that supports scar formation while attenuating osseous tissue formation.

In other aspects, DBM is deactivated or partially deactivated by chemical or heat treatment of BMP-2 to denature the BMP-2 protein. The osteoinductive response of DBM can be attenuated to a level where the DBM supports heterotopic cartilage formation, but does not support osseous tissue formation. Partially deactivating DBM using chemicals or heat prior to implantation in the IVD results in the formation of cartilage without subsequent bone formation. Illustratively, extraction of DBM with about four (4) M guanidine hydrochloride or about six (6) M urea for a period of time ranging from about two (2) hours to about twelve (48) hours removes from about fifty percent (10%) to about ninety percent (100%) of the growth factor content of the DBM.

In other embodiments, incubating mineralized bone particles in phosphate buffer at about thirty-seven degrees Celsius (37° C.) for time periods ranging from about four (4) hours to about seventy-two (72) hours allows proteases contained within the mineralized bone to specifically destroy BMPs. When the bone is eventually demineralized to produce DBM, the BMP content is reduced creating a scaffold that supports scar formation and cartilage formation.

Further embodiments include mechanical stabilization of the disc space in combination with treatment using deactivated DBM. Thus, in some aspects, granulation tissue and/or scar tissue is removed from the IVD of a damaged or degenerated disc. In other aspects, complete discectomy is performed. In either case, a scaffold is introduced into the resulting space and the bioactive component comprising deactivated DBM is added to the scaffold. The scaffold can be made of any suitable material, including, for example, metal or polymers. An exemplary material is polyetheretherketone (PEEK), although other non-degradable materials may be used including, without limitation, other polyetherketones, polyamide, polyaryletherketone polyimide, hydrogels, silicone rubbers, and the like.

In some embodiments, a resorbable material may be used for the scaffold or implant. In this manner, the resorbable material operates to support the vertebrae while fibrous formation occurs, but resorbs to leave only the fibrous material in the disc space. Resorbable materials that may be used include, but are not limited to polyhydroxyacids, copolymers of lactide and glycolide of various compositions, polyamino acids, degradable polymers, and other materials.

In other embodiments, the scaffold or implant has motion preserving characteristics. For example, the implant may be a dynamic stabilization device which fits at least partially in the disc space. The device would allow for some motion at the disc site, while inducing scar formation according to one or more embodiments disclosed herein. The dynamic stabilization device could be a ball and detent device, a device made from flexible materials or rigid materials formed in a flexible configuration, or other possible constructs.

Any scaffold suitable for insertion into the disc space while keeping the annulus intact and distracting and maintaining controlled motion at the disc space is contemplated herein. Likewise, any scaffold that supports the space created by a full discectomy including annulus removal is contemplated herein. It is also helpful if the scaffold facilitates delivery of a chondrogenic biologic agent (such as, for example, deactivated DBM). Existing PEEK interbody fusion cages are suitable for some embodiments described herein. In some embodiments, one or more Breckenridge™ Interbody devices available from Lanx, Inc. of Broomfield, Colo. may be used as the interbody or scaffold.

### Methods For Inducing Fibrosis In A Joint In Need of Repair

Embodiments of the invention provide methods for inducing fibrosis in a joint or on a bone surface in need of repair. For purposes of the present disclosure “fibrosis” refers to the formation of fibrous connective tissue on one or more bone surfaces in a joint in need of repair or in damaged articular cartilage in a joint in need of repair. The formation of fibrous connective tissue using embodiments of the invention is enhanced as compared to formation of fibrous connective tissue in the absence of embodiments of the invention, i.e., formation of fibrous connective tissue for the joint surface in need of repair under normal physiologic conditions for the patient and in the absence of added fibrosis inducing agents.

In one embodiment, a health care professional or agent determines a type of fibrosis inducing agent(s); determines the requirement, if any, of grinding the bone surface in the joint space during administration of the fibrosis inducing agent; determines the need of MSC or fibroblast cell implantation into the joint in need of repair; and determines the need for angiogenesis inducing factors. In some embodiments the health care professional may additionally determine that mechanical disruption of an adjacent bone surface may be warranted to enhance the release of intravascular products in the environment of the joint in need of repair.

In addition the health care professional may determine that a carrier or implant material is also required, including collagen, hydrogels, reabsorbable polymer materials, and other like materials.

The health care professional or agent then provides the desired combination of factors above to a patient having a joint in need of repair. The combination is provided or administered in a manner to facilitate fibrosis in the joint in need of repair, and therapeutic result is charted for a time sufficient to ensure the health professional's desired outcome. Outcome may be followed by symptom ablation, fibrosis detection via MRI, X-ray, or other scanning methodology or by an optical or other like technique. Additional therapeutic treatments may be required on any one patient to ensure proper fibrosis of a joint in need of repair in accordance with the present invention. Additional treatments may be the same or include new combinations to ensure that each patient obtain the best possible result.

### EXAMPLES

The following examples are provided for illustrative purposes only and are not intended to limit the scope of the invention.

### Example 1

A patient presents with significant low back pain unresponsive to conservative measures. The work-up identifies advanced degenerative disc disease at one level with an associated collapse of the disc space. The patient is felt to be a candidate for treatment using the embodiments of the invention. Because of the disc space collapse, optimal treatment consists of disc space distraction to elevate the disc to its healthy height followed by accelerating fibrosis of the intradiscal space. The patient is taken to the OR and a fenestrated Fernstrom ball is placed to elevate disc height and release the fibrosing factor. In this case PLA is released via the Fernstrom ball. After the surgery is completed the patient is instructed to limit activities until fibrosis has been completed in 30-45 days. An analysis of the patient indicates that the injured disc has been substantially replaced with fibrotic tissue.

### Example 2

A patient presents with significant cartilage loss in a joint. The ends of the target joint are ground down and a fibrosis inducing agent is introduced into the joint in conjunction with a collagen implant to facilitate scar formation on the bone surface. The patient is instructed to limit activities until fibrosis is completed in 30-45 days. An analysis of the patient indicates that the damaged area of the joint is substantially covered with fibrotic tissue. The fibrous connective tissue generated provides a flexible and hydrated tissue similar to undamaged cartilage.

### Example 3

DBM is produced from ovine bone using processes identical to those used to manufacture human DBM. Briefly, tibial and femoral diaphyseal segments are cleaned of soft tissue and marrow. The cleaned bone is ground to a powder about one hundred (100) to about five hundred (500) microns in size in a freezer mill. Ground particles are defatted in 40 volumes of 100% ethanol for 4 hours and dried under a fume hood overnight. The bone powder is demineralized in 40 volumes of 0.6N HCl for 30 minutes. The acid is replenished and the demineralization process is allowed to continue for 60 minutes. The demineralized bone powder is washed with deionized water until the eluate exceeds pH 4.0, then lyophilized.

Lyophilized SPP-24 is ground into a fine powder and combined with DBM. A 50-fold (w/w) excess of SPP-24 completely ablates the effects of BMP-2. However, concentrations of growth factors in DBM vary by lot and preparation methodology. For example, Wildemann et al. reports BMP-2 concentrations of 0.004 g BMP-2 per gram total protein. Wildemann et al. (2007) Quantification of various growth factors in different demineralized bone matrix preparations. J. of Biomed. Mat. Res. Part A. 81A(2): 437-442. Thus, SPP to DBM ratios range from about 1:10 to about 2:1, for example, about 1:10, about 1:8, about 1:7, about 1:6, about 1:5, about 1:4, about 1:3, about 1:2, about 1:1, and about 2:1.

### Example 4

Ovine tibial and femoral diaphyses are cleaned of soft tissue and marrow and ground to a particle size of about 100 microns to about 500 microns in a freezer mill. The ground bone is washed with about 100 volumes of cold water for about 2 hours and then defatted with about 50 volumes of 100% ethanol for 4 hours. Excess ethanol is removed and the residual alcohol allowed to evaporate overnight under a fume hood.

Partial inactivation of the osteoinductive capacity of bone is achieved by incubation of cleaned and washed ground bone at 25° C. in 100 mM phosphate buffer, pH 7.0, for several hours. The bone preparations are then demineralized by treatment with 40 volumes of 0.6 N HCl at 23° C. for two 30 minute periods. Excess acid is washed away from bone with 40 volumes of deionized water six times for 5 minutes. The DBM preparation is subsequently lyophilized.

### Example 5

Ovine marrow stromal cells (MSC) are obtained by aspiration from the iliac crest of euthanized sheep using 16-gauge needles. Heparinized aspirates are diluted 1:3 with phosphate-buffered saline (PBS) and filtered through a 70 μm nylon strainer. Sterile DBM preparations (SPP-inhibited, heat-inactivated, and untreated DBM control) are seeded with ovine MSC and maintained in α-modified MEM (ccMEM) supplemented with 10% fetal bovine serum (FBS) and 1% Penn/Strep under standard culture conditions. Media is changed every 2-3 days. Cell viability is investigated after day 1 and day 4 of culture using the Alamar Blue assay. On day 7 of culture half of the cells are supplemented with an osteogenic differentiation media consisting of αMEM supplemented with 10% FBS, 1% Penn/Strep, Dexamethasone (10⁻⁸M final concentration), ascorbic acid (50 μg/mL final), and β-glycerol phosphate (8 mmol final). Assays performed at 3, 7, and 14 days after introduction of osteogenic differentiation media include a dimethylmethylene blue (DMMB) photospectrometric assay for glycosaminoglycan (GAG) content and an alkaline phosphatase (ALP) assay for bone formation. Total collagen accumulation is quantified by HPLC-based amino acid analysis of hydroxyproline.

The DMMB and collagen assays demonstrate production of cartilaginous extracellular matrix (ECM) components including GAG and collagen in standard media. The ALP assay indicates reduction/quenching of ECM mineralization when challenged by osteogenic differentiation media.

### Example 6

The ovine model allows for the use of currently-available human instrumentation for the interbody spacer, and skeletally mature sheep are a well-accepted lumbar spinal model.

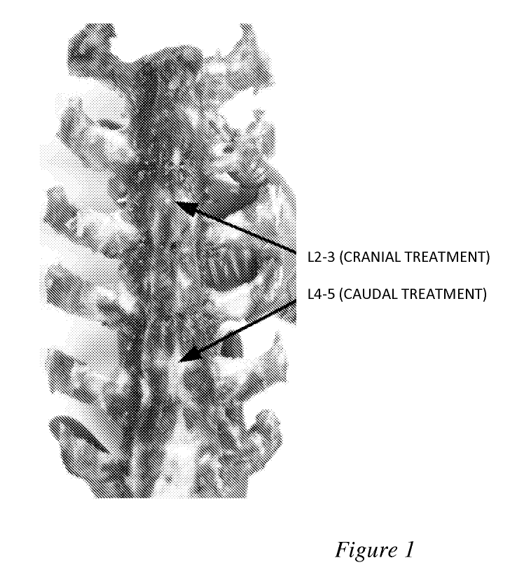

Surgical procedure: All procedures are performed with the approval of the Institutional Animal Care and Use Committee and based upon experimental design protocols that have been approved in the past. Mature female sheep undergo a procedure similar to anterior lumbar interbody fusion with a PEEK cage using a left anterior retroperitoneal approach. Treatments are randomized among the following groups, n=6 per group: autograft (positive control), heat-inactivated DBM, DBM +spp24, sham surgery (negative control). Each animal receives one control and one experimental treatment in separate discs. In some cases, one unoperated level is maintained between treated discs (FIG. 1). Sheep are monitored per standard post-operative care protocols. Two (2) sheep from each group are humanely euthanized at 2-week, 6-week, and 12-week time-points; time-points are chosen to correspond to expected peak fibrous tissue content, peak GAG content, and recovery of mineral content in the autograft group.

Radiography: Plain radiographs are taken under general anesthesia immediately post-operatively and after euthanasia. Radiographs are analyzed for disc height changes and correlated to histological findings.

Histology: Explanted spines are trimmed in the transverse plane cranial and caudal to the operated levels and placed in 10% neutral buffered formalin in preparation for undecalcified histology. After fixation, the specimens are dehydrated in graded solutions of ethyl alcohol over the course of approximately 4 weeks. The samples are cleared with acetone and sequentially infiltrated in graded catalyzed methyl methacrylate. After polymerization into a hardened plastic block histological sections are taken in the sagittal plane.

Sections are differentially stained to allow detection of bone, cartilage, and collagen within the section. Evaluation includes qualitative histopathological assessment of inflammation, fibrosis, angiogenesis, and chondrogenesis as well as histomorphometric quantification of newly-formed tissue in the intervertebral space. Tissue sections are immunostained for types I and II collagen and for GAG with alcian blue. To examine the mechanism of repair, tissue is also immunostained with antisera to TGF-β1 and TGF-β2.

Experimentally treated discs show generation and maintenance of fibrocartilaginous bridging tissue through week 12, and an absence of mineralization. Experimentally treated discs also show maintenance of disc height as indicated by post operative and euthanasia radiographs.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

The specification contains numerous citations to references such as patents, patent applications, and scientific publications. Each is hereby incorporated by reference for all purposes.

What is claimed is:

1. A method of treatment on a joint in need of cartilage repair comprising:
performing a distraction of space between adjacent bone surfaces in the joint, the distraction sufficient to provide sufficient space height between the adjacent bone surfaces to facilitate fibrosis by a fibrosis inducing agent; and
contacting a surface within the joint with an amount of the fibrosis inducing agent to induce fibrosis on the surface, wherein induced fibrosis in the joint facilitates repair of the joint.

2. The method of claim 1 wherein the distraction is performed using a non-resorbable spacer.

3. The method of claim 1 wherein the distraction is temporary and accomplished with a reabsorbable material.

4. The method of claim 1 further comprising introducing autologous stem cells to a site in the joint in need of repair.

5. The method of claim 1 further comprising introducing autologous fibroblasts to a site in the joint in need of repair.

6. The method of claim 1 wherein the fibrous inducing agent comprises partially inactivated demineralized bone matrix (DBM).

7. A method of treatment of an intervertebral disc in need of repair, comprising:
administering into a portion of the intervertebral disc in need of repair an amount of deactivated demineralized bone matrix (DBM) sufficient to induce fibrosis in the disc, wherein induced fibrosis in the disc facilitates repair of the disc.

8. The method of claim 7, wherein the DBM is deactivated through treatment with an inhibitor of BMP.

9. The method of claim 8, wherein the inhibitor is selected from the group consisting of alpha-2-HS glycoprotein, noggin, chordin, DAN, sclerostin and secreted phosphoprotein 24 (SPP-24).

10. The method of claim 8, wherein the inhibitor is SPP-24.

11. The method of claim 7 wherein the DBM is deactivated through a heat treatment.

12. The method of claim 7 wherein the DBM is deactivated through a chemical treatment.

13. The method of claim 7 further comprising performing a distraction of space at the intervertebral disc in need of repair, wherein the distraction is sufficient to provide sufficient space height at the intervertebral disc to facilitate fibrosis by the deactivated DBM.

14. The method of claim 13, wherein the distraction is performed using a mechanical spacer.

15. A system for treatment of an intervertebral disc in need of repair comprising:
deactivated demineralized bone matrix (DBM) in an amount sufficient to induce fibrosis in the disc while attenuating osteogenic tissue formation, wherein induced fibrosis in the disc facilitates repair of the disc; and
a scaffold.

16. The system of claim 15, wherein the scaffold comprises a polyetheretherketone interbody spacer.

17. A kit for treatment of an intervertebral disc in need of repair comprising:
a polyetheretherketone interbody spacer; and
instructions for administering into a portion of the intervertebral disc an amount of deactivated demineralized bone matrix (DBM) sufficient to induce fibrosis in the disc, wherein induced fibrosis in the disc facilitates repair of the disc.

18. A method for inducing fibrocartilaginous tissue in an intervertebral disc in need of repair while attenuating osseous tissue formation, the method comprising administering into a portion of the intervertebral disc in need of repair an amount of deactivated demineralized bone matrix (DBM) sufficient to induce fibrosis in the disc, wherein induced fibrosis in the disc facilitates repair of the disc.

19. A method of treating an intervertebral disc between two adjacent vertebrae, the method comprising:
performing at least a partial discectomy on the intervertebral disc;
inserting an implant into the intervertebral disc, the implant having a height corresponding to at least a percentage of a desired spacing between the adjacent vertebrae; and
administering into a portion of the intervertebral disc an amount of partially deactivated demineralized bone matrix (DBM) sufficient to induce fibrosis in the intervertebral disc, the partially deactivated DBM adapted to promote fibrous tissue formation and attenuate bony tissue formation.

20. The method of claim 19 further comprising preparing the deactivated DBM prior to the administering into the portion of the intervertebral disc, the preparing of the deactivated DBM comprising treatment of DBM with heat, chemical(s), or an inhibitor of BMP.
